(19) United States
(12) Patent Application Publication
(10) Pub. No.: US 2026/0306822 A1
(43) Pub. Date: Oct. 8, 2026

(54) Co-Molded Rigid Cap With Integrated RFID and Cap End Torque Transmission Feature

(71) Applicant: Becton Dickinson France, Le Pont-de-Claix (FR)

(72) Inventors: Simon Rempfer, St Hilaire de la Cote (FR); Lukasz Dolega, St Martin Le Vinoux (FR); Senturk Ozturk, Echirolles (FR); Jérémy Zucchelli, St Martin d'Heres (FR); Jérémie Vaxelaire, Ambilly (FR)

(21) Appl. No.: 19/481,548

(22) PCT Filed: May 28, 2024
(86) PCT No.: PCT/EP2024/064649

§ 371 Date: Nov. 5, 2025

(30) Foreign Application Priority Data
May 30, 2023 (EP) 23305855.1

(51) Int. Cl. A61M 39/20; A61M 5/178; G06K 19/07

(52) U.S. Cl. CPC A61M 39/20; A61M 5/178; G06K 19/0723; A61M 2205/60

(57) ABSTRACT

Provided herein is a rigid cap including an inner molded portion having a proximal end and a distal end, with the proximal end having an open end and including a female connector formed thereon and the distal end having a closed end. The rigid cap also includes an RFID element positioned over the inner molded portion at the distal end so as to extend about at least a portion of the distal end and an outer molded portion formed over the inner molded portion and over the RFID element at the distal end, so as to encase the RFID element between the inner molded portion and the outer molded portion, with the outer molded portion having a proximal end and a distal end. The inner molded portion and/or the outer molded portion includes a plurality of circumferentially spaced apart teeth extending out axially from the distal end thereof.

### CROSS-REFERENCE TO RELATED APPLICATION

The present application claims priority to European Application No. 23305855.1 entitled “Co-Molded Rigid Cap with Integrated RFID and Cap End Torque Transmission Feature” filed May 30, 2023, the entire disclosure of which is hereby incorporated by reference.

### BACKGROUND OF THE INVENTION

### Field of the Invention

The present disclosure relates generally to rigid caps, such as for syringes, and more particularly to rigid caps with an integrated RFID tag and torque transmission feature formed thereon.

### Description of Related Art

Syringes are used in a variety of environments for administering liquids, such as medications or drugs, to a patient. Many syringes are provided as prefilled syringes, which provide the convenience of rapidly delivering the liquid therein to a patient without the need to first aspirate the medication from another container and meter its volume. For dispensing fluids, the prefilled syringe will typically include a syringe barrel with an opening at a distal end adapted for fluid communication with a patient (e.g., a luer-type fitting (needleless access device) fitted at the distal end of the syringe barrel and connected to a fluid line of a patient) and a plunger assembly inserted through the opposite proximal end of the barrel. The plunger assembly typically includes an elongated plunger rod extending out of the barrel, and a plunger head or stopper disposed at the distal end of the plunger rod. The stopper or plunger stopper is typically made of elastomeric material, and comprises a body with a tail disposed at its proximal end adapted for attachment to the distal end of the plunger rod, and a head disposed at its distal end. On an external cylindrical wall of the body is defined a plurality of annular outwardly protruding ribs adapted to ensure the container closure integrity of a syringe when the stopper is inserted into the syringe.

In using prefilled syringes, it is desirable to label the prefilled syringe to include data concerning the syringe and/or the contents (i.e., medication) included in the syringe and, in some embodiments, to enable tracking of the syringe. That is, it is desired to have the ability to track the history of individual syringes or lots of syringes from early in the manufacturing life through to the end use of the device. Identification and/or tracking of the syringe may be provided via use of an electronic element that contains data thereon and that can be conventionally read in and read out in a contactless fashion. As one example, an electronic element such as, for example, an RFID tag, may be incorporated into the syringe to provide identification and tracking for the syringe. In some known syringes, the RFID tag is incorporated into a rigid cap that is positioned over the opening of the syringe barrel on the distal end, such as a cap that mates with a luer-type fitting at the distal end of the syringe barrel. The RFID tag may be embedded within the rigid cap to provide protection thereto, with such embedding of the cap achieved via a co-molding process for forming the rigid cap.

While it is known to integrate an RFID tag into a rigid cap to provide identification and/or tracking of a syringe, existing rigid caps with integrated RFID tags may suffer from issues associated with the functionality of the rigid cap. As one example, it is known that batches of syringes are typically packaged, stored, and shipped in storage containers known as “nests,” and that the sizing/diameter of the rigid cap may need to be controlled in order to enable machine automated placement and/or withdrawal of syringes into and/or out from the nest without interference. However, the integration of an RFID tag within a rigid cap via a co-molding process can lead to challenges in controlling the cap diameter, while still achieving desired functionality with the cap. Specifically, it is desired to include splines (“spline teeth”) on the outer surface of the rigid cap that extend radially outward and by which the cap can be gripped and twisted via an associated twisting tool, in order to secure the cap onto the syringe, but inclusion of the RFID tag can reduce the space available for such spline teeth to be formed on the rigid cap. Accordingly, the radial height of the spline teeth is reduced in order to keep the rigid cap below a required dimensional limit, and this reduction in the spline height and the amount it protrudes radially outward from an adjacent cap outer surface can have a negative impact on external torque transmission from a twisting tool to the rigid cap when securing the cap to the syringe.

Accordingly, a need exists in the art for a rigid cap that incorporates an RFID tag therein but that includes features thereon that provide for efficient external torque transmission.

### SUMMARY OF THE INVENTION

Provided herein is a rigid cap for use with a syringe. The rigid cap includes a cap housing comprising a proximal end and a distal end and defining an internal cavity, and an RFID element integrated with the cap housing at the distal end. The cap housing includes a plurality of circumferentially spaced apart teeth extending out axially from the distal end thereof.

In certain configurations, the cap housing includes a first molded portion, with the RFID element integrated on an inner surface of the first molded portion.

In certain configurations, the RFID element is a hard tag including an RFID chip and antenna encapsulated in a rigid sleeve, washer, or disc.

In certain configurations, the cap housing includes a first molded portion, with the RFID element integrated on an outer surface of the first molded portion.

In certain configurations, the RFID element is an over-molded tag with in-mold electronics.

In certain configurations, the cap housing includes a first molded portion and a second molded portion, the second molded portion integrated over the first molded portion and over the RFID element at the distal end, so as to encase the RFID element between the first molded portion and the second molded portion.

In certain configurations, the RFID element is a flat RFID inlay that conforms to a cylindrical outer surface of the first molded portion.

In certain configurations, the RFID inlay includes a first edge and an opposing second edge, with the RFID inlay extending partially about the distal end of first molded portion, such that a gap is present between the first and second edges, and wherein the second molded portion is in direct contact with the first molded portion at a location of the gap.

In certain configurations, the plurality of circumferentially spaced apart teeth are provided on each of the first molded portion and the second outer portion.

In certain configurations, the plurality of teeth on the first molded portion is aligned circumferentially with and joined to the plurality of teeth on the second molded portion.

In certain configurations, a distal end of the first molded portion has a first radial thickness comprised between 0.2 mm and 1.0 mm, and preferably between 0.5 mm and 0.8 mm, and the second molded portion has a second radial thickness comprised between 0.2 mm and 1.0 mm, and preferably between 0.5 mm and 0.8 mm.

In certain configurations, each of the plurality of teeth has a height of between 0.5 and 3.0 mm.

In certain configurations, each of the plurality of teeth comprises a rectangular, triangular, curved, or wavy shaped tooth having a flat distal end.

In certain configurations, the plurality of teeth comprises between two teeth and sixteen teeth, and preferably between four teeth and eight teeth.

Also provided herein is a syringe that includes a syringe barrel having a proximal end and a distal end and defining a chamber, with the syringe barrel having an opening at the proximal end. The syringe also includes a plunger assembly inserted through the opening and axially movable within the chamber of the syringe barrel, with the plunger assembly including a plunger rod having an elongated body and extending between a proximal end and a distal end, and a stopper attached to the distal end of plunger rod and positioned within the barrel chamber. The syringe further includes a rigid cap having a cap housing with a proximal end and a distal end and defining an internal cavity, and an RFID element integrated with the cap housing at the distal end. The cap housing includes a plurality of circumferentially spaced apart teeth extending out axially from the distal end thereof. The rigid cap is coupled to an adapter at the distal end of the syringe barrel.

### BRIEF DESCRIPTION OF THE DRAWINGS

FIG. 1 is a perspective view of a syringe, with which embodiments of the disclosure are implemented;

FIG. 2 is an exploded view of the syringe of FIG. 1;

FIG. 3 is a perspective view of a rigid cap included in syringe of FIG. 1, according to a non-limiting embodiment described herein;

FIG. 4 is a side cross-sectional view of the rigid cap of FIG. 3, shown coupled to a male connector of a syringe;

FIG. 5 is a perspective view of an inner molded portion of the rigid cap of FIG. 3, with an RFID tag positioned thereon;

FIG. 6 is a perspective view of a rigid cap included in syringe of FIG. 1, according to another non-limiting embodiment described herein;

FIG. 7 is a perspective view of an inner molded portion of the rigid cap of FIG. 6, with an RFID tag positioned thereon;

FIG. 8 is a perspective view of a rigid cap included in syringe of FIG. 1, according to another non-limiting embodiment described herein;

FIG. 9 is a perspective view of an inner molded portion of the rigid cap of FIG. 8, with an RFID tag positioned thereon;

FIG. 10 is a perspective view of a rigid cap included in syringe of FIG. 1, according to another non-limiting embodiment described herein;

FIG. 11 is a perspective view of an inner molded portion of the rigid cap of FIG. 10, with an RFID tag positioned thereon;

FIG. 12 is an exploded perspective view of a rigid cap included in syringe of FIG. 1, according to another non-limiting embodiment described herein;

FIG. 13 is a side cross-sectional view of a rigid cap included in syringe of FIG. 1, according to another non-limiting embodiment described herein;

FIG. 14 is a side cross-sectional view of a rigid cap included in syringe of FIG. 1, according to another non-limiting embodiment described herein; and

FIG. 15 is a perspective view of a rigid cap included in syringe of FIG. 1, according to another non-limiting embodiment described herein.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

In the present disclosure, the distal end of a component or of a device means the end furthest away from the hand of the user and the proximal end means the end closest to the hand of the user, when the component or device is in the use position, i.e., when the user is holding a syringe in preparation for or during use. Similarly, in this application, the terms “in the distal direction” and “distally” mean in the direction toward the distal tip of the syringe, and the terms “in the proximal direction” and “proximally” mean in the direction opposite the direction of the distal tip of the syringe.

Aspects and embodiments of the disclosure are directed to a rigid cap with an integrated RFID tag and torque transmission feature formed thereon. The rigid cap is formed as a co-molded cap with the RFID tag embedded therein between distinct molded portions or layers of the cap. The rigid cap includes a front or distal end feature formed thereon that enables external torque applied to the cap, such as via a process twisting or screwing tool engageable with the cap, to be transmitted through the cap for twisting engagement of the cap with an associated connection on a syringe barrel.

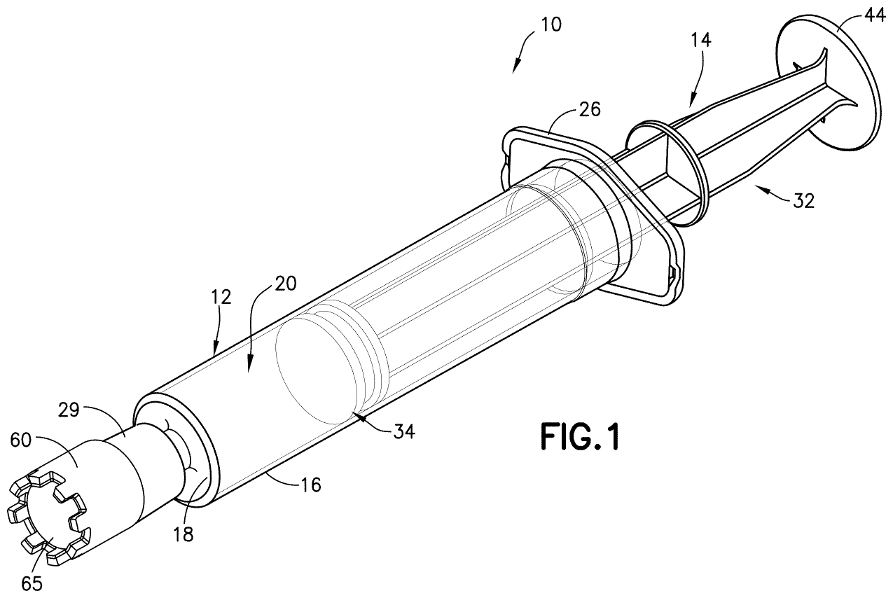

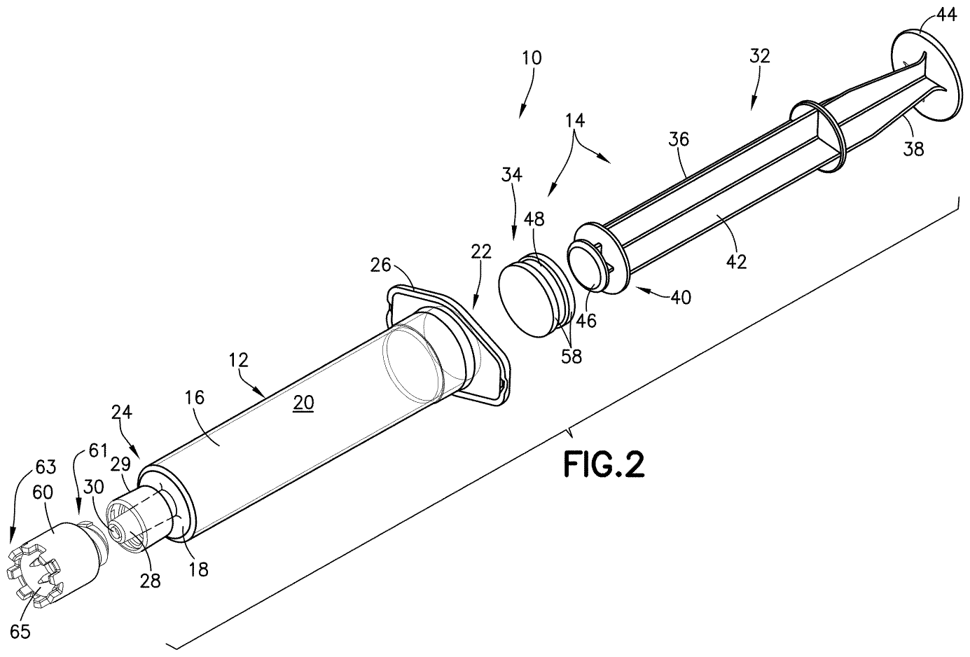

Referring to FIGS. 1 and 2, shown is a non-limiting embodiment of a syringe 10, such as a prefilled syringe for example, with which aspects or embodiments of the disclosure may be implemented. The syringe 10 generally includes a syringe barrel 12 and a plunger assembly 14. The plunger assembly 14 is axially movable within the syringe barrel 12 to an advanced position to facilitate administering of an injectable fluid (e.g., medication) to a patient, for example.

The syringe barrel 12 is formed of a generally cylindrical outer wall 16 and an end wall 18 that collectively define a chamber 20 for retaining fluid therein. The syringe barrel 12 includes an open proximal end 22 configured to receive the plunger assembly 14 therein and a distal end 24 at which end wall 18 is positioned. The proximal end 22 of the syringe barrel 12 may include a flange 26 to facilitate handling and positioning of the syringe 10 and to maintain the relative position of the syringe barrel 12 with respect to the plunger assembly 14 during medication administration. At the distal end 24, a tip 28 extends distally outward from the end wall 18 and defines a lumen 30 in fluid communication with the chamber 20. A luer lock adapter 29 is mounted on an external surface of the tip 28 that is coupleable to a rigid cap 60 that covers the tip 28 and/or to a corresponding needle hub (not shown) to which the syringe 10 is to be brought into engagement for administering medication to a patient. The adapter 29 comprises a cylindrical body having a threaded inner surface 33 that is configured to engage an outer surface of a threaded portion of the rigid cap 60, as explained in further detail below. While a specific adapter 29 is shown and described herein, it is recognized that adapters of various constructions known in the art may be provided on syringe 10 for connecting the syringe 10 to a corresponding needle hub for expelling the injectable material into a target location.

The plunger assembly 14 of syringe 10 is formed of an elongate plunger rod 32 (more generally “plunger 32,” as used hereafter) and a plunger head or stopper 34. The plunger 32 may include a main body 36 extending between a plunger proximal end 38 and a plunger distal end 40. In some embodiments, the main body 36 may include a plurality of elongate vanes or walls 42 extending axially along a length thereof between the plunger proximal end 38 and the plunger distal end 40. A thumb press 44 is positioned at the plunger proximal end 38 that may be engaged by a thumb (or other finger) of the user to apply a distally directed force to the plunger assembly 14 to move the plunger 32 with respect to the syringe barrel 12. In some embodiments, a flanged extension member 46 (e.g., disc-shaped flange) is positioned at the plunger distal end 40 that is configured to mate with the stopper 34.

The stopper 34 of plunger assembly 14 is positioned at the plunger distal end 40 so as to be movable along with the plunger 32 within the chamber 20 of syringe barrel 12. The stopper 34 may be made from a material that is different from the material of the plunger 32 and that is capable of forming a tight seal with the syringe barrel 12 as it is advanced therethrough. In some embodiments, the stopper 34 includes a receptacle (not shown) formed therein that is sized and configured to receive the flanged extension member 46 of plunger 32, with the flanged extension member 46 coupling with the receptacle via a press fit connection, for example, to secure the stopper 34 to the plunger 32, although it can be appreciated that the attachment member 48 and plunger distal end 40 can be secured by other techniques known in the art. The attachment member 48 may further include a pair of spaced-apart annular flanges 58 formed thereon (i.e., O-rings) that form a tight seal with the cylindrical outer wall 16 of syringe barrel 12.

As shown in FIGS. 1 and 2, syringe 10 further includes a rigid cap 60 that may be coupled to the adapter 29 of syringe barrel 12 to protect the tip 28 from contamination and/or maintain the chamber 20 in a sealed condition or state prior to use of the syringe 10. The rigid cap 60 may be configured as a rigid cap that mates with the adapter 29, so as to provide a fluid-tight seal over the tip 28, with a proximal end 61 of the rigid cap 60 configured to engage the adapter 29 and a distal end 63 (i.e., a “front end”) configured to be closed with an elastomeric inner cap 65 (“RiTC”) to form a closed rigid cap 60 that covers the tip 28.

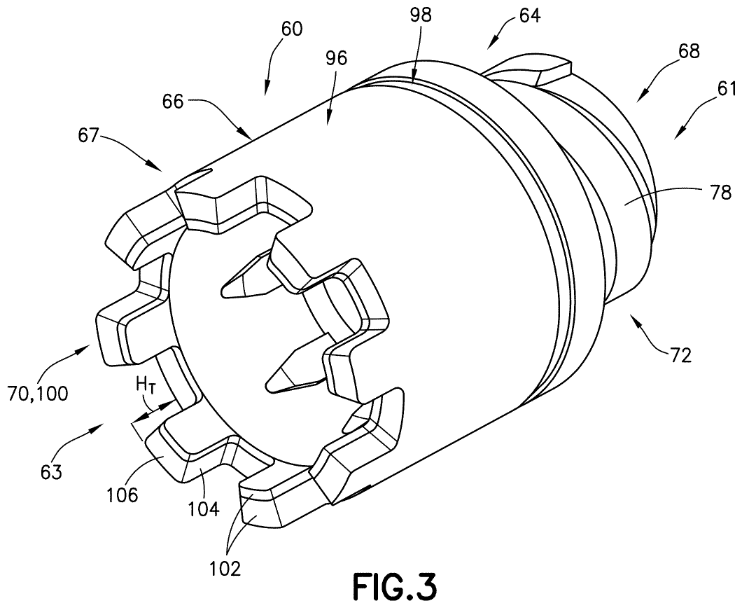

According to aspects and embodiments of the disclosure, and as will be shown and described in further detail below in FIGS. 3-15 according to various aspects of the disclosure, the rigid cap 60 includes an RFID element or tag 62 integrated therein that allows for identification and/or tracking of the syringe 10. That is, the RFID element 62 may include or store information thereon regarding the contents of the syringe 10 (i.e., the medication or drug included therein, if a prefilled syringe) and the manufacturing history of the syringe and/or may provide location information to an associated reader, so as to enable tracking of the syringe 10. According to embodiments, the RFID element 62 may be provided on an inner or outer surface of a portion of the rigid cap 60 or may be embedded within the rigid cap 60, as will be explained in further detail below.

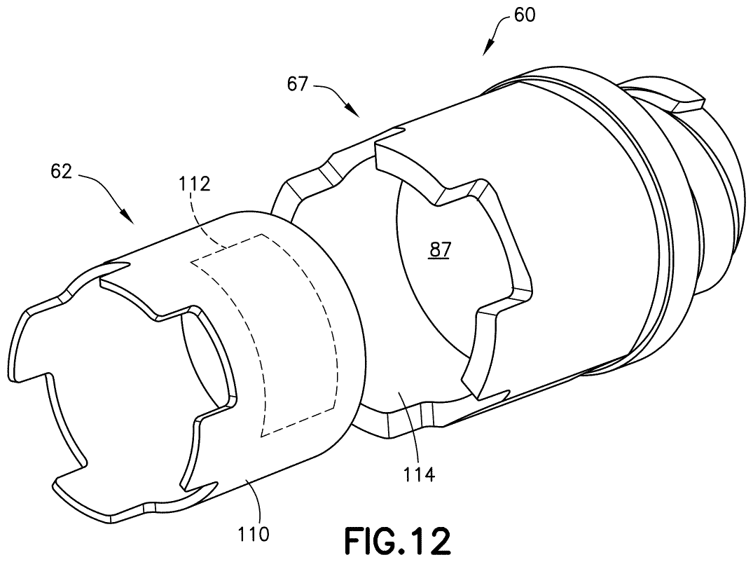

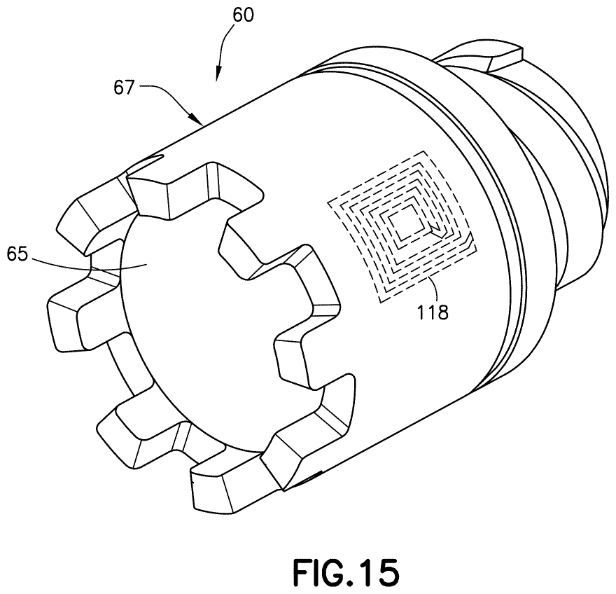

According to embodiments, the RFID element 62 may be provided in any of a number of forms that enables integration thereof with the rigid cap 60. In some embodiments, the RFID element 62 may be an inlay including a chip or integrated circuit (IC), antenna, and substrate (i.e., film face) (see FIGS. 3-11). In other embodiments, the RFID element 62 may be a hard tag that includes a chip and antenna encapsulated in a rigid sleeve, washer, or disc (see FIGS. 12-14). In other embodiments, the RFID element 62 may be a printed tag that includes an antenna directly printed on a 3D part with a bonded chip, or an overmolded tag with in-mold electronics technology (see FIG. 15).

According to embodiments, it is recognized that the RFID element 62 may be any type of tag available to the skilled person. That is, those of skill in the art will clearly understand that other tags different than RFID may also be used. For instance, the tag may be a Low Frequency Radio Frequency Identification (LF-RFID) tag (i.e., frequencies from 30 KHz to 300 KHz). The RFID tag may also be a High Frequency Radio Frequency Identification (HF-RFID) tag (i.e., frequencies from 1 MHz to 15 MHz), an Ultra High Frequency Radio Frequency Identification (UHF-RFID) tag (i.e., frequencies from 400 MHz to 1,000 MHz), or a double frequency tag including simultaneously a HF-NFC and an UHF RFID. Alternatively, the RFID tag may be a High-Frequency Near Field Communication (HF-NFC) tag (i.e., frequencies from 1 MHz to 15 MHZ).

A rigid cap 60 is shown in further detail in FIGS. 3-5 according to one aspect or embodiment of the disclosure. The rigid cap 60 is formed as a rigid cap that is formed by a molding process, such as an injection molding process. When the rigid cap 60 comprises a first molded portion 64 (an “inner molded portion”) and a second molded portion 66 (an “outer molded portion”), the rigid cap 60 is formed as a co-molded cap that is formed via two separate molding processes-with the inner molded portion 64 formed during a first molding shot or process and the outer molded portion 66 formed during a second molding shot or process. Each of the inner and outer molded portions 64, 66 is thus formed of a material suitable for molding, such as polypropylene, polyethylene, polyvinylchloride, polystyrene, polycarbonate, acrylonitrile butadiene styrene, styrene acrylonitrile, or similar polymers, or a combination thereof. The inner and outer molded portions 64, 66 may collectively define a cap housing 67 of the rigid cap 60. As explained further below, in manufacturing of the rigid cap 60, the second molded portion 66 may be formed over part of the inner molded portion 64, with the RFID element 62 integrated between the inner and outer molded portions 64, 66.

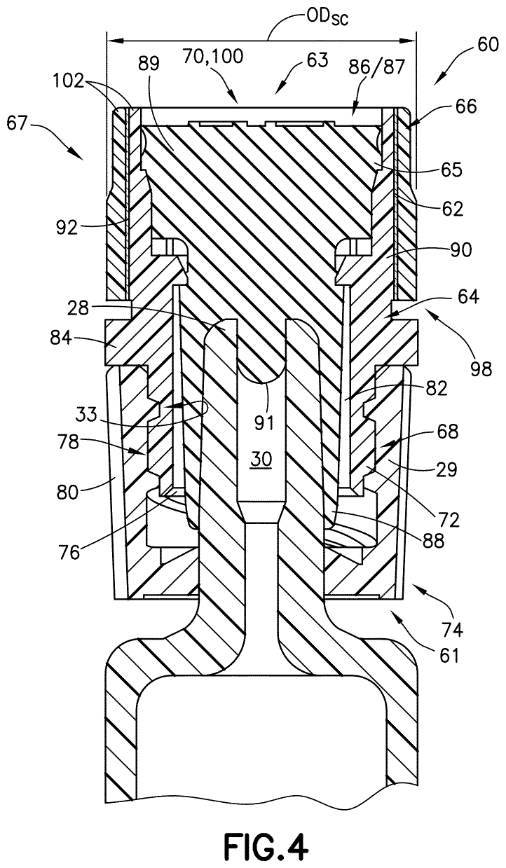

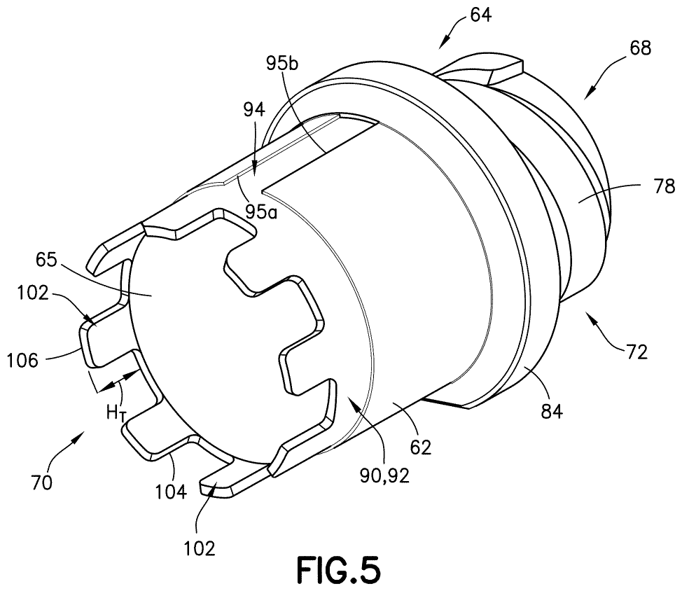

As best shown in FIGS. 4 and 5, the inner molded portion 64 of rigid cap 60 may generally be defined as including a proximal end 68 and a distal end 70. The proximal end 68 is configured as an open end and includes a connecting portion 72 formed thereon suitable for coupling to the adapter 29 or syringe 10. As previously described, in some embodiments the distal end 24 of the syringe barrel 12 includes a tip 28 thereon on which an adapter 29 is secured/mounted and, in such embodiments, the connecting portion 72 is configured to mate with the adapter 29. The connecting portion 72 may include a cylindrical portion 76 having a threaded outer surface 78 configured to engage the threaded inner surface 33 of adapter 29, with the cylindrical portion 76 defining an opening 82 within which a portion of the elastomeric inner cap 65 may be inserted.

In some embodiments, an annular flange 84 may be formed on the inner molded portion 64 adjacent to the cylindrical portion 76 of the connecting portion 72. The annular flange 84 is positioned distally from the proximal end 68 of inner molded portion 64 (and between the proximal end 68 and the distal end 70) such that, upon engagement of the threaded outer surface 78 of connecting portion 72 with the threaded inner surface 33 of the adapter 29, the annular flange 84 will abut a distal end of the adapter 29, thereby providing a sealed engagement between the rigid cap 60 and the syringe barrel 12.

The distal end 70 of inner molded portion 64 is configured as an open end that is configured to receive therein the elastomeric inner cap 65, which seals off the distal end 70 of rigid cap 60. That is, the inner molded portion 64 may be formed to include cylindrical wall 90 that defines a circular opening 86 at the distal end 70 thereof, with the inner cap 65 mounted in the opening 86 and extending into a cavity 87 defined by the inner molded portion 64.

The inner cap 65 may be made of a rubber material such as styrene butadiene rubber or isopren bromobuytyl blend, or a thermoplastic elastomer. A (rubber) formulation of the inner cap 65 is selected to be inert to a medical solution to be filled into the syringe 10, with sufficient heat resistance to undergo the necessary sterilization procedures, and with suitable gripping and releasing properties for slipping the inner cap 65 on the tip 28 of the syringe 10 and releasing it after use. The inner cap 65 can be manufactured by a molding process such as injection molding.

The inner cap 65 comprises a proximal skirt 88 having a hollow cylindrical shape which may be slightly tapered in order to fit with the tapered shape of the tip 28 of syringe 10, and a distal sealing base 89 which can present a central protrusion or nipple 91 surrounded by said skirt 88, forming an inner cavity having a substantially circular shape adapted to engage the tip 28 of the syringe 10. The central protrusion 91 extends proximally from the distal sealing base 89 and is centered with respects to this distal sealing base. The central protrusion 91 can slightly penetrate into the distal end of the tip 28 of the syringe 10 (i.e., into lumen 30). The inner diameter of the skirt 88 is chosen so that the skirt is slightly stretched radially when mounted on the tip 28 of the syringe 10. In other words, the inner diameter of the skirt 88 is slightly smaller than the outer diameter of the tip 28 of the syringe 10. In this way, the inner wall of the skirt 88 tightly encloses the outer surface of the tip 28 of the syringe 10. The tight geometry and the elastomeric material enable the inner cap 65 to provide a liquid-tight sealing of the tip 28 of the syringe 10, avoiding any leak and/or contamination of medical solution.

In the embodiment of the rigid cap 60 shown in FIGS. 3-5, the RFID element 62 of rigid cap 60 is applied or set onto the outer surface 92 of the sidewall 90 at the distal end 70 of inner molded portion 64. According to embodiments, the RFID element 62 conforms to the cylindrical outer surface 92 of the sidewall 90 and may wrap at least substantially thereabout. In some embodiments, the RFID element 62 is configured to wrap a majority of the way around sidewall 90, but not entirely thereabout, such that a gap 94 is present between a first edge 95a and an opposing second edge 95b of the RFID element 62. According to embodiments, the RFID element 62 may be glued onto the cylindrical outer surface 92 of the sidewall 90 or bonded with the outer surface 92 via cohesion with melt plastic, to aid in retaining the RFID element 62 in place.

As shown in FIGS. 3 and 4, after molding of inner molded portion 64 and application of the RFID element 62 onto the distal end 70 of the inner molded portion 64, the outer molded portion 66 may be formed (i.e., molded) over the inner molded portion 64 and over the RFID element 62 to complete fabrication of the rigid cap 60. The outer molded portion 66 is formed over the inner molded portion 64 and over the RFID element 62 at the distal end 70 of the inner molded portion 64, so as to encase the RFID element 62 between the inner molded portion 64 and the outer molded portion 66. The outer molded portion 66 is formed as a generally tube-shaped member that surrounds the distal end 70 of inner molded portion 64, with the outer molded portion 66 having a generally cylindrical outer surface 96 thereon. The outer molded portion 66 includes a proximal end 98 that, in some embodiments, is positioned adjacent the annular flange 84, and a distal end 100 that is adjacent and/or generally aligned with the distal end 70 of inner molded portion 64. According to embodiments, the outer molded portion 66 and the inner molded portion 64 may each have a radial thickness between 0.2 mm and 1.0 mm, and preferably between 0.5 mm and 0.8 mm. In some embodiments, the outer molded portion 66 is formed to have a radial thickness that is greater than the radial thickness of the distal end 70 of the inner molded portion 64. Also, in embodiments where the RFID element 62 wraps only partially around sidewall 90, the outer molded portion 66 will be in direct contact with the inner molded portion 64 at the location of gap 94, so that a bond is formed between the inner and outer molded portions 64, 66.

In accordance with aspects of the disclosure, it is recognized that an outer diameter of the rigid cap 60, outer diameter ODsc, may be constrained based on packaging requirements of the syringe 10. That is, it is known that batches of syringes 10 are typically packaged, filled, stored, and/or shipped in nests having numerous chimneys formed therein to hold the syringes. Removal of the syringes 10 from this nest is performed by axially sliding the syringes 10 relative to the nest and, if a rigid cap 60 exceeds a pre-determined diameter, the syringe 10 may become lodged within a chimney during extraction from the nest. Consequently, an automated processing line associated with the syringes 10, such as for automatically filling the syringes 10 with medication/drugs for example, may need to be stopped until the syringe(s) 10 are able to be dislodged from the nest, leading to undesirable downtime and maintenance costs, as well as potential damage to the syringe(s) 10. The embedding of RFID element 62 between the inner and outer molded portions 64, 66 in rigid cap 60 increases the outer diameter ODsc.

In use of rigid cap 60, it is recognized that it is desirable to include features thereon that assist with threadingly engaging the rigid cap 60 with the adapter 29 (i.e., mating the threaded outer surface 78 of connecting portion 72 with the threaded inner surface 33 of adapter 29). While such features are commonly provided as splines that are spaced circumferentially about the outer surface 96 of outer molded portion 66 and that extend out radially therefrom-and that enable use of a twisting tool (i.e., process screwing tool) that engages the splines to apply a screwing torque to the rigid cap 60—-the inclusion of such splines may be inhibited based on the diameter constraints of the rigid cap 60, with this especially true with the embedding of RFID element 62 between the inner and outer molded portions 64, 66 in rigid cap 60 and the increase in diameter of the cap resulting therefrom. In some cases, a maximum dimension of the outer diameter ODsc may be approximately 10.9 mm.

Responsive to the above, aspects and embodiments of the disclosure are directed to the inclusion of a plurality of end teeth 102 on the rigid cap 60 that extend axially out from the distal end 63 of the cap 60 (from distal end 63 of housing 67), i.e., extend out in a longitudinal direction L of the rigid cap 60. Similar to the splines described above, the teeth 102 provide for engagement of a twisting tool with the rigid cap 60 and for the twisting tool to apply a screwing torque to the cap 60. However, the teeth 102 are formed on the rigid cap 60 such that they do not extend radially out past the outer surface 96 of the outer molded portion 66, but rather provide for engagement of a twisting tool with the rigid cap 60 at the distal end 63 (front end) thereof.

As shown in FIGS. 3 and 4, in accordance with one aspect or embodiment of the disclosure, each of the inner molded portion 64 and the outer molded portion 66 includes a plurality of teeth 102 formed at the distal end thereof 70, 100 and that extend out axially out therefrom. The teeth 102 are spaced about the circumference of the inner molded portion 64 and outer molded portion 66, with some embodiments having the teeth 102 spaced equidistantly from one another. The teeth 102 on the inner molded portion 64 are aligned circumferentially with the teeth 102 on the outer molded portion 66, such that the teeth 102 on the inner and outer molded portions 64, 66 overlap and are joined with one another when formed as part of the molding processes.

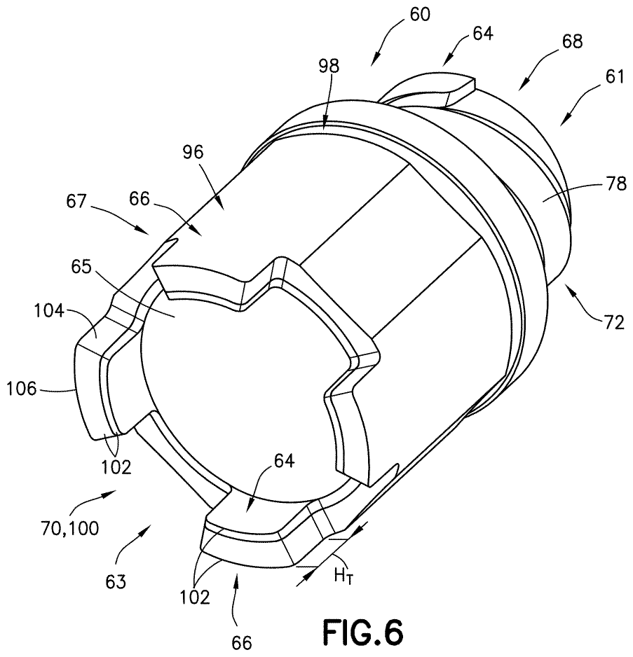

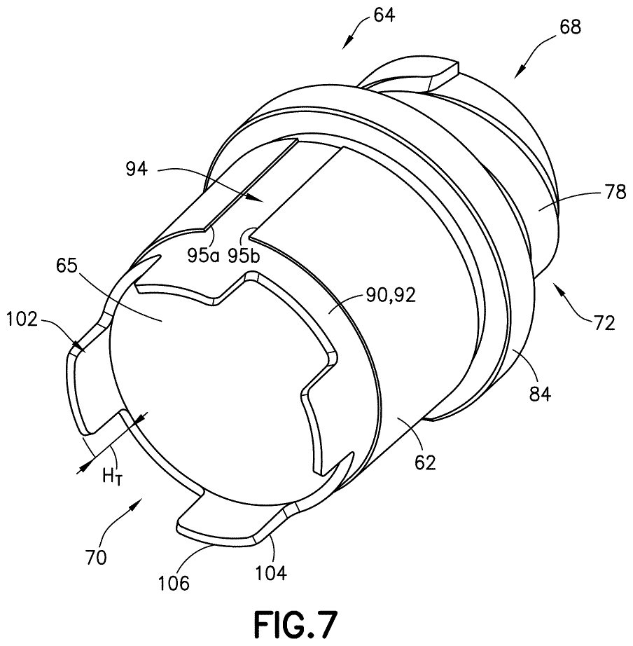

According to aspects of the disclosure, the number of teeth 102 included on rigid cap 60 may vary, with the number of teeth 102 provided being determined (at least in part) by the configuration of a twisting tool to be employed for securing the rigid cap 60 to the syringe barrel 12. In the embodiment of FIGS. 3-5, eight (8) teeth 102 are formed on the inner and outer molded portions 64, 66, but it is recognized that a greater or lesser number of teeth 102 could be formed. For example, in the embodiment of FIGS. 6 and 7, four (4) teeth 102 are formed on the inner and outer molded portions 64, 66. In other embodiments, the number of teeth 102 included on rigid cap 60 may be anywhere from two (2) to sixteen (16) teeth 102.

In use of the rigid cap 60, the teeth 102 are configured to provide a front end engagement feature or surface with which a twisting tool (i.e., process screwing tool) may engage to apply a screwing torque to the rigid cap 60 when securing the cap to the syringe barrel 12—i.e., when engaging the threaded outer surface 78 of the connecting portion 72 on inner molded portion 64 with the threaded inner surface of adapter 29 on syringe barrel 12. In some embodiments, the teeth 102 provide a castellated front end surface with which the twisting tool may engage-with each of the teeth 102 having a generally rectangular shape where side edges 104 of the teeth 102 extend out orthogonally from the distal end 70, 100 of the inner and outer molded portions 64, 66 and a flat distal edge 106 is arranged orthogonal to the side edges 104. Each of the teeth 102 may have a height (i.e., length), HT, of between 0.5 and 3.0 mm by which the teeth 102 extend out from the second end 70, 100, in order to provide for proper engagement between the twisting tool and the teeth 102 and enable a desired transfer of screwing torque from the twisting tool to the teeth 102 and the rigid cap 60. In other embodiments, each of the teeth 102 may have a triangular, curved, or wavy shape with a flat distal edge 106.

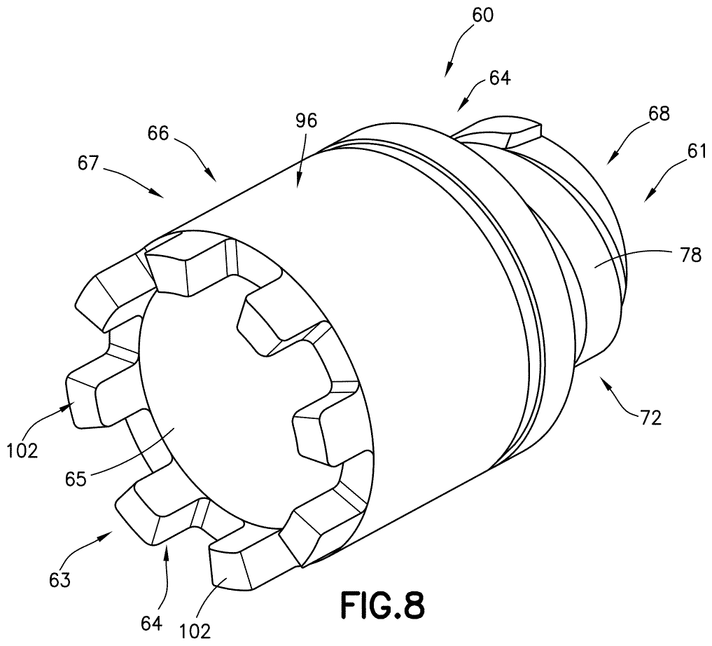

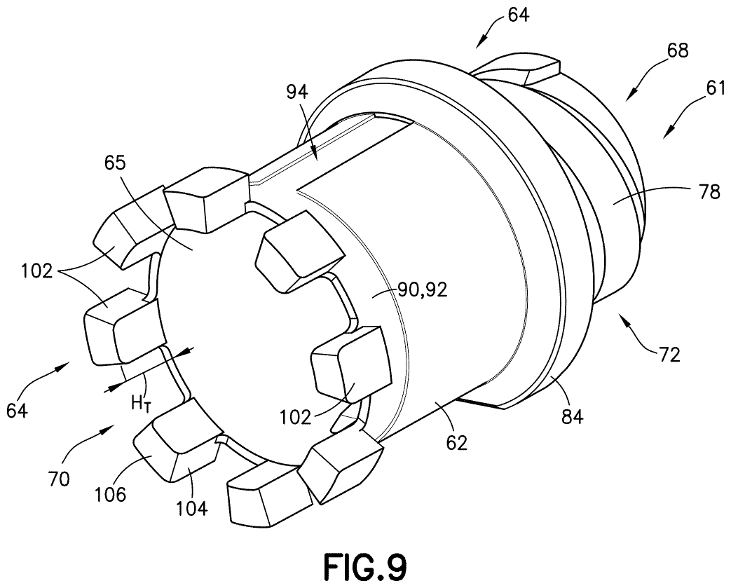

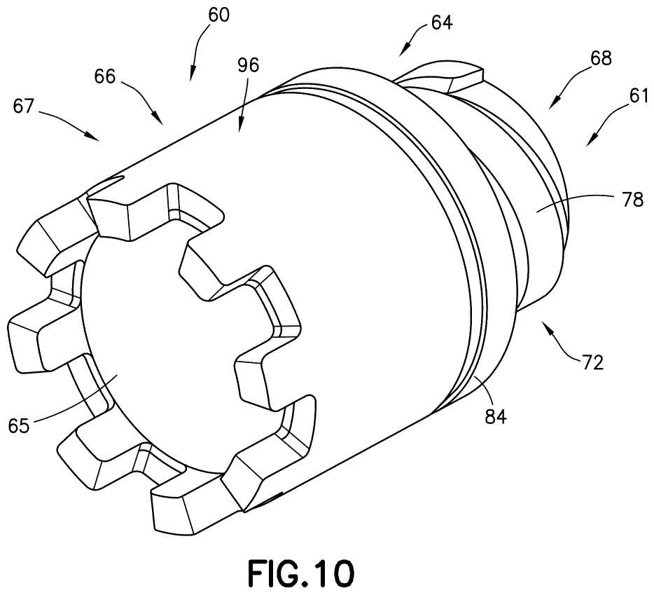

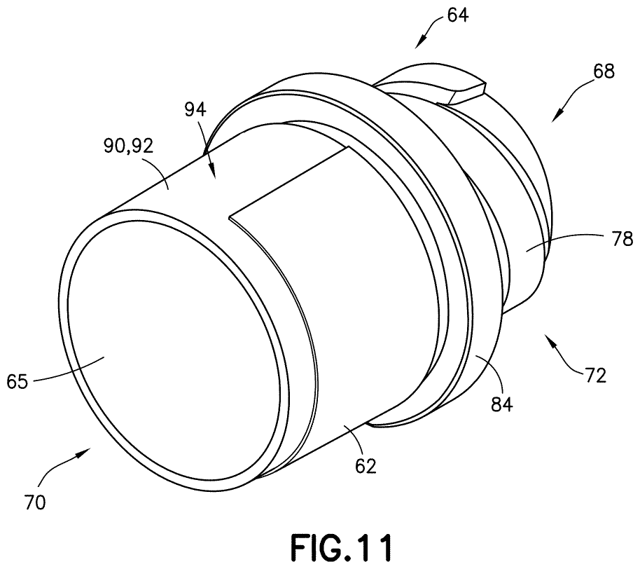

While the embodiments of rigid cap 60 shown in FIGS. 3-7 show a plurality of teeth 102 formed on each of the inner molded portion 64 and the outer molded portion 66, it is recognized that only one of the inner molded portion 64 and outer molded portion 66 may have teeth 102 formed thereon, according to additional embodiments. In one example, and as shown in FIGS. 8 and 9, the teeth 102 are formed only on inner molded portion 64—i.e., formed as part of a first-shot molding of the inner molded portion 64. In another example, and as shown in FIGS. 10 and 11, the teeth 102 are formed only on outer molded portion 66—i.e., formed as part of a second-shot molding of the outer molded portion 66. In each of these embodiments, the thickness of the teeth 102 may be increased as compared to the remainder of the inner molded portion 64 (i.e., of second end 70 thereof) or the outer molded portion 66 of which the teeth 102 form a part of. The thickness of the teeth 102 should be such that they resist breaking when engaged by a twisting tool and when a screwing torque is applied via the twisting tool.

Furthermore, while the embodiments of rigid cap 60 shown in FIGS. 3-11 are shown and described as being formed via a co-molding process and including an inner molded portion 64 and outer molded portion 66, it is recognized that the rigid cap 60 (i.e., housing 67 of the rigid cap 60) may be formed as a single, integral component (i.e., formed via a single molding process). As indicated above, in embodiments where the housing 67 of rigid cap 60 is formed as a singular molded component, the RFID element 62 may be integrated on an inner surface or an outer surface of the housing 67.

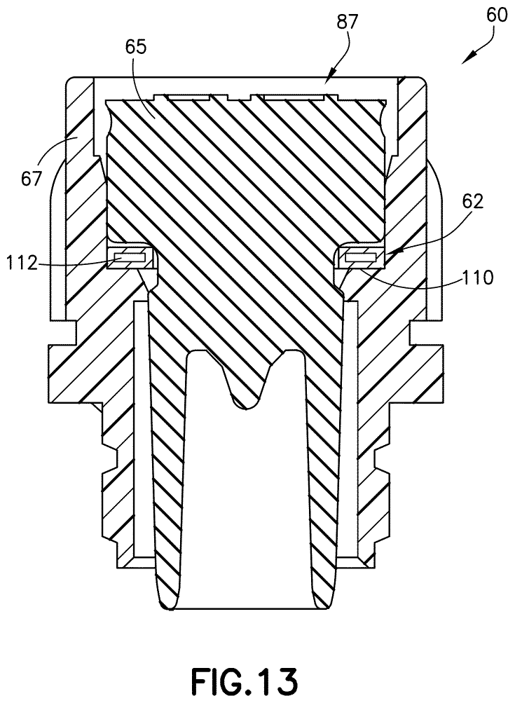

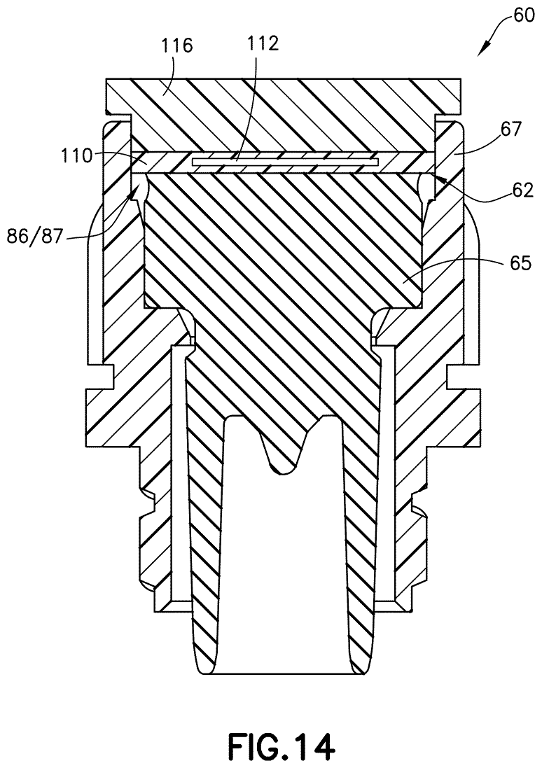

Referring now to FIGS. 12-14, embodiments of rigid cap 60 are provided where the RFID element 62 is located on an inner surface of an integrally formed/molded housing 67 with the RFID element 62 provided as a hard tag (“hard tag 62”) that includes a rigid housing member 110 and an encapsulated RFID tag 112. In the embodiment of FIG. 12, the rigid housing member 110 of the hard tag 62 is constructed as a rigid sleeve that is positioned within the cavity 87 of housing 67 and is secured to an inner surface 114 of the housing 67 surrounding cavity 87, such as by being glued onto the inner surface 114, secured with the inner surface 114 via a snap-fit feature, or mounted in-force against the inner surface 114 (i.e., a press or interference fit). While hard tag 62 is shown in FIG. 12 as including a rigid housing member 110 with teeth formed thereon, it is recognized that rigid housing member 110 may instead be formed as a cylindrical member (with only housing 67 having teeth 102 thereon). In the embodiment of FIG. 13, the rigid housing member 110 of the hard tag 62 is constructed as a washer-shaped member (with RFID tag 112 embedded therein) positioned at the bottom of cavity 87 and about inner cap 65, with the inner cap 65 securing the hard tag 62 in place relative to rigid cap 60. In the embodiment of FIG. 14, the rigid housing member 110 of the hard tag 62 is constructed as a disc-shaped member (with RFID tag 112 embedded therein) positioned at the top of cavity 87 and at a distal end of inner cap 65. In some embodiments, a protective cover 116 is fit into opening 86 at the distal end of housing 67 protecting the hard tag 62 and securing it in place relative to rigid cap 60.

Referring now to FIG. 15, an embodiment of rigid cap 60 is provided where the RFID element 62 is located on an outer surface of an integrally formed/molded housing 67. In one embodiment, and as shown in FIG. 15, the RFID element 62 may be configured as a directly printed antenna and bonded chip 118 provided directly on the outer surface of the housing 67. In other embodiments, the RFID element 62 is provided as an over-molded tag 118 with in-mold electronics provided on the outer surface of the housing 67.

Beneficially, embodiments of the invention thus provide a rigid cap with an integrated RFID tag and external torque transmission feature formed thereon. The rigid cap is formed as a co-molded cap with the RFID tag embedded therein between distinct inner and outer molded portions or layers of the cap. The rigid cap includes a plurality of axially extending front end teeth formed thereon that enable engagement of a twisting tool with the cap, so as to provide for transmission of an external torque through the cap for performing a twisting engagement of the cap with an associated connection on a syringe barrel. As the teeth extend out axially from the front/distal end of the rigid cap rather than radially out from an outer surface, the external dimensions of the rigid cap may be controlled while still enabling embedding of an RFID tag therein.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

1. A rigid cap for use with a syringe, the rigid cap comprising:
a cap housing comprising a proximal end and a distal end and defining an internal cavity; and
a radio frequency identification (RFID) element integrated with the cap housing at the distal end;
wherein the cap housing comprises a plurality of circumferentially spaced apart teeth extending out axially from the distal end thereof, wherein the teeth extend in a longitudinal direction of the cap and provide for engagement with a twisting tool to apply a torque thereto.

2. The rigid cap of claim 1, wherein the cap housing comprises a first molded portion, with the RFID element integrated on an inner surface of the first molded portion.

3. The rigid cap of claim 1, wherein the RFID element comprises a hard tag including an RFID chip and antenna encapsulated in a rigid sleeve, washer, or disc, and wherein the hard tag is positioned within the internal cavity of the cap housing and secured to an inner surface thereof.

4. The rigid cap of claim 1, wherein the cap housing comprises a first molded portion, with the RFID element integrated on an outer surface of the first molded portion.

5. The rigid cap of claim 4, wherein the RFID element comprises an over-molded tag with in-mold electronics.

6. The rigid cap of claim 1, wherein the cap housing comprises a first molded portion and a second molded portion, the second molded portion integrated over the first molded portion and over the RFID element at the distal end, so as to encase the RFID element between the first molded portion and the second molded portion.

7. The rigid cap of claim 6, wherein the RFID element comprises a flat RFID inlay that conforms to a cylindrical outer surface of the first molded portion.

8. The rigid cap of claim 7, wherein the RFID inlay comprises a first edge and an opposing second edge, with the RFID inlay extending partially about the distal end of first molded portion, such that a gap is present between the first and second edges, and wherein the second molded portion is in direct contact with the first molded portion at a location of the gap.

9. The rigid cap of claim 6, wherein the plurality of circumferentially spaced apart teeth are provided on each of the first molded portion and the second outer portion.

10. The rigid cap of claim 9, wherein the plurality of teeth on the first molded portion is aligned circumferentially with and joined to the plurality of teeth on the second molded portion.

11. The rigid cap of claim 6, wherein a distal end of the first molded portion has a first radial thickness comprised between 0.2 mm and 1.0 mm, and preferably between 0.5 mm and 0.8 mm, and the second molded portion has a second radial thickness comprised between 0.2 mm and 1.0 mm, and preferably between 0.5 mm and 0.8 mm.

12. The rigid cap of claim 1, wherein each of the plurality of teeth has a height of between 0.5 and 3.0 mm.

13. The rigid cap of claim 1, wherein each of the plurality of teeth comprises a rectangular, triangular, curved, or wavy shaped tooth having a flat distal end.

14. The rigid cap of claim 1, wherein the plurality of teeth comprises between two teeth and sixteen teeth, and preferably between four teeth and eight teeth.

15. A syringe, comprising:
a syringe barrel comprising a proximal end and a distal end and defining a chamber, the syringe barrel having an opening at the proximal end;
a plunger assembly inserted through the opening and axially movable within the chamber of the syringe barrel, the plunger assembly including:
a plunger rod comprising an elongated body and extending between a proximal end and a distal end; and
a stopper attached to the distal end of plunger rod and positioned within the barrel chamber; and
a rigid cap, the rigid cap comprising:
a cap housing comprising a proximal end and a distal end and defining an internal cavity, and
a radio frequency identification (RFID) element integrated with the cap housing at the distal end;
wherein the cap housing comprises a plurality of circumferentially spaced apart teeth extending out axially from the distal end thereof, wherein the teeth extend in a longitudinal direction of the cap and provide for engagement with a twisting tool to apply a torque thereto:
wherein the rigid cap is coupled to an adapter at the distal end of the syringe barrel.
